(19) United States
(12) Patent Application Publication
(10) Pub. No.: US 2013/0251662 A1
(43) Pub. Date: Sep. 26, 2013

(54) COMPOSITION FOR THE PERMANENT SHAPING OF HUMAN HAIR

(71) Applicants: Jonathan Wood, Weinheim (DE); Alexandra Meuser, Egelsbach (DE); Jörg Schneider, Griesheim (DE)

(72) Inventors: Jonathan Wood, Weinheim (DE); Alexandra Meuser, Egelsbach (DE); Jörg Schneider, Griesheim (DE)

(73) Assignee: KAO GERMANY GMBH, Darmstadt (DE)

(21) Appl. No.: 13/990,336

(22) PCT Filed: Nov. 29, 2011
(86) PCT No.: PCT/EP2011/071246

§ 371 Date: May 29, 2013

(30) Foreign Application Priority Data
Dec. 1, 2010 (EP) 10015164.6

(51) Int. Cl. A61K 8/84; A61K 8/81; A61Q 5/04

(52) U.S. Cl. CPC A61K 8/84; A61Q 5/04; A61K 8/8182; A61K 8/817
USPC 424/70.51; 424/70.5

(57) ABSTRACT

The present invention concerns a composition for the permanent shaping of human hair based on at least one reducing agent and further comprising a first cationic polymer consisting of a copolymer of vinylpyrrolidone and vinylimidazole and a homopolymer of dimethyl diallyl ammonium chloride and a second cationicpolymer selected from urea copolymers and process for permanent shaping human hair with the said compositions.

### SUMMARY

The present invention concerns a composition for the permanent shaping of human hair used both for the permanent waving of human hair with an excellent waving effect as well as for the straightening of either naturally or chemically curled hair.

It is generally known that permanent waving is carried out in two steps, the reductive splitting of the cysteine disulfide bonds in the hair by a reducing agent and the subsequent neutralization by application of an oxidizing agent, whereby the cysteine disulfide bonds are restored.

The reducing agent still most frequently used today is thioglycolic acid, also in form of the salts thereof, in particular its ammonium salt, although numerous other thio compounds have been proposed for this purpose, which, however, mostly did not succeed.

The compositions containing thioglycolates are customarily applied at a pH-value between 7 and 10, in particular 8.5 and 9.5.

Such compositions vary in their waving and/or straightening performance and, therefore, there is still need for further improvement, especially in permanent shaping of damaged to strongly damaged hair and in particular for permanent shaping hair streaks including parts with various damage level in length.

Use of polymers in permanent shaping compositions for hair has been known for many years. For examples in EP 797861 A1 permanent shaping compositions are disclosed comprising homopolymer of dimethyl diallyl ammonium chloride. In an earlier application which has not been published yet for our company, permanent shaping compositions have been disclosed comprising copolymer of vinylpyrrolidone and quaternized vinylimidazole.

The present invention starts from the task of providing a composition for the permanent shaping of human hair with excellent waving and straightening performance. Hair waved or straightened with composition disclosed herein looks and feels natural upon touching by hand. For waved hair it is especially important that the hair has excellent elasticity and bounce.

Accordingly, the first object of the present invention is a composition for permanent shaping and/or straightening hair based on at least one reducing agent further comprising a first cationic polymer consisting of a copolymer of vinylpyrrolidone and vinylimidazole and homopolymer of dimethyl diallyl ammonium chloride and a second cationic polymer selected from urea copolymers. Vinylimidazole in the copolymer of first cationic polymer is not quaternised. Cationic charge density of the first cationic polymer is preferably between 3 and 4 mEq/g and particularly 3.7 mEq/g at pH 7.0.

Recently a cationic polymer has become commercially available under the trade name Luviquat Sensation from BASF with CTFA name Polyquaternium-87 which is the cationic polymer particularly suitable as the first cationic polymer for the compositions of the present invention.

Composition of the present invention comprises preferably a first cationic polymer at a concentration of 0.1 to 2.5%, preferably 0.2 to 2%, more preferably 0.25 to 1.5% and most preferably 0.25 to 1% by weight calculated to total composition.

Composition of the present invention comprises further a second cationic polymer selected from urea copolymers. Suitable and most preferred cationic polymer is Polyquaternium-2 commercially available under the trade name Mirapol A-15 from Rhodia.

Second cationic polymer is preferably comprised in the compositions of the present invention at a concentration of 0.1 to 2.5%, preferably 0.2 to 2%, more preferably 0.25 to 1.5% and most preferably 0.25 to 1% by weight calculated to total composition.

It has furthermore been find out that weight ratio of the first ad second cationic polymers play an important role in obtaining the best permanent shaping efficiency. In preferred embodiment of the present invention, first and second cationic polymers are comprise in the composition at a first to second cationic polymer ratio in the range of 5:1 to 1:5, preferably 3:1 to 1:3 and more preferably 2:1 to 1:2 and most preferably 1:1.

It is furthermore preferred that total concentration of the first and the second cationic polymers in the compositions is in the range of 0.2 to 5%, preferably 0.2 to 2%, more preferably 0.25 to 1.5% and most preferably 0.25 to 1% by weight calculated to total composition.

The permanent shaping compositions according to the invention comprise at least one reducing compound at a concentration of at least 0.5% by weight calculated to total composition. Preferred are thioglycolic acid and thiolactic acid as well as the salts thereof, in particular the ammonium and ethanolamine salts. Further useful thio compounds are in particular cysteine or the hydrochloride thereof, homocysteine, cysteamine, N-acetyl cysteine, thioglycerol, ethanediol monothioglycolate, 1.2-propyleneglycol monothioglycolate (see also WO-A 93/1791), 1-3-propanediol monothioglycolate or the isomer mixture resulting therefrom, 1.3-butanediol and 1.4-butanediol monothioglycolate and the isomer mixtures therefrom, polyethylene glycol, such as di-, tri- and tetraethyleneglycol monothioglycolates, glycerol monothiolactate and further thio acids and the esters thereof, as well as mixtures thereof.

The use of inorganic reducing sulfur compounds such as sodium hydrogen sulfite is basically also possible.

The total reduction agent content in the compositions according to the invention customarily amounts from 0.5 to 15%, preferably 1.0 to 12.5%, more preferably 2.0 to 12.5% by weight, calculated to total composition as free thioglycolic acid as reference substance.

The permanent shaping compositions containing reducing agents can, if necessary, comprise alkalizing agents. Their quantity is dependent on the reducing agent and the desired pH-value of the composition. Reducing agent compositions preferably contain 0.1% to 5%, in particular 0.5% to 2.5% by weight thereof, calculated to the total composition. Alkalizing agents preferred within the scope of the invention are ammonium carbamate, ammonia and/or ammonium(bi)carbonate, monoethanolamine and triethanolamine. It is desirable to adjust the pH-value between about 6.5 and 10.5, preferably about 7 to 9.5.

Permanent shaping compositions according to the invention are suited for use both for the permanent waving, i.e. curling of human hair and for the straightening, i.e. smoothing thereof.

The viscosity best suited for the permanent shaping compositions according to the invention proved to be in the range of 1 to 10,000 mPa·s, preferably about 1 to about 5,000 mPa·s, measured at 20° C. in a Brookfield viscosimeter (no. 5 spindle), whereas the viscosity suited for the straightening compositions is preferably higher in a range up to 50,000 mPa·s, preferably up to 30,000 mPa·s measured at 20° C. in a Brookfield viscosimeter (no. 5 spindle).

The viscosity is adjusted by addition of the appropriate amounts of thickening agents known per se, such as cellulose derivatives. Thickening may as well be realized by formulating a composition in form of an emulsion with the use of C₁₀-C₂₂-fatty alcohols, in mixture with long mono alkyl chain quaternary ammonium surfactants.

Permanent shaping compositions according to the present invention preferably comprise surfactants selected from anionic, nonionic, cationic and amphoteric ones. Their proportion ranges from 0.05% to 10%, in particular from 0.1% to 5% by weight, calculated to total composition.

Suitable anionic surfactants are especially the known alkyl ether sulfates and carboxylic acids, in particular in form of their alkali salts, as well as protein fatty acid condensates.

Suitable nonionic surfactants, which are preferred within the scope of the invention, are in particular C₈-C₁₈-fatty alcohol polyglycol ethers, fatty acid polyglycol esters, fatty acid alkanolamides, amineoxides, and especially C₈-C₁₈-alkyl polyglucosides.

Also possible is the incorporation of amphoteric surfactants, such as the known alkyl betaines, alkyl amido betaines, and alkyl amphoacetates.

Further according to a further preferred embodiment, permanent shaping compositions comprise at least one cationic surfactant according to general formula

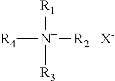

where R₁s a saturated or unsaturated, branched or non-branched alkyl chain with 8-24 C atoms or

R₅CONH(CH₂)ₙ

where R₅ is saturated or unsaturated, branched or non-branched alkyl chain with 7-21 C atoms and n has typical value of 0-4 or

R₆COO(CH₂)ₙ

where R₆ is saturated or unsaturated, branched or non-branched alkyl chain with 7-21 C atoms and n has typical value of 0-4, and

R₂ is a saturated or unsaturated, branched or non-branched alkyl chain with 1-24 C atoms or

R₅CONH(CH₂)ₙ

where R₅ is saturated or unsaturated, branched or non-branched alkyl chain with 7-21 C atoms and n has typical value of 0-4 or

R₆COO(CH₂)ₙ

where R₆ is saturated or unsaturated, branched or non-branched alkyl chain with 7-21 C atoms and n has typical value of 0-4,
and R₃ and R₄ are same or different lower alkyl chain with 1 to 4 carbon atoms which may be substituted with one or two hydroxyl group, and X is chloride, bromide or methosulfate.

Concantration of cationic surfactant is in the range from 0.05% to 5%, preferably 0.1% to 2.5% by weight, calculated to total composition.

Suitable long-chain quaternary ammonium compounds are in particular cetyl trimethyl ammonium chloride, dimethyl dicetyl ammonium chloride, trimethyl cetyl ammonium bromide chloride, stearyl trimethyl ammonium chloride, dimethyl stearyl benzyl ammonium chloride, benzyl tetradecyl dimethyl ammonium chloride, dimethyl dihydrogenated tallow ammonium chloride, lauryl pyridinium chloride, lauryl dimethyl benzyl ammonium chloride, lauryl trimethyl ammonium chloride, tris-(oligooxyethyl)alkyl ammonium phosphate, cetyl pyridinium chloride, etc.

Further permanent shaping compositions of the present invention may comprise additional cationic polymer. Basically suitable are all cationic polymers listed under the generic name “Polyquaternium” in the CTFA International Cosmetic Ingredient Dictionary. Examples are Polyquaternium 4, Polyquaternium 5, Polyquaternium 6, Polyquaternium 7, Polyquaternium 10, Polyquaternium 11, Polyquaternium 16, Polyquaternium 22 and Polyquaternium 28, and Polyquaternium 39,

The cationic polymers also include the quaternized products of graft polymers from organopolysiloxanes and polyethyl oxazolines described in EP-A 524 612 and EP-A 640 643. Such polymer is known with its CTFA name Polysilicone-9.

Concantration of one or more additional cationic polymers is in the range from 0.05% to 2.5%, preferably 0.1% to 1.5% by weight, calculated to total composition.

Permanent shaping compositions of present invention can comprise additionally at least one organic solvent. Suitable organic solvents are 2-methyl-1,3-propanediol, mono and dialcohols or the ethers thereof, in particular mono-C₁-C₃-alkyl ether, ethanol, n-propanol, isopropyl alcohol, 1-methoxypropanol, 1-ethoxypropanol and ethoxydiglycol, diols and their esters 1,3- and 1,4-butanediol, diethyleneglycol and the monomethyl and monoethyl ether thereof, dipropylene glycol and the monomethyl and monoethyl ether thereof, glycerol, hexanetriol, ethyl carbitol, benzyl alcohol, benzyloxy ethanol, propylene carbonate, N-alkyl pyrrolidone, and urea or their mixture preferably in an amount from about 0.1% to 10% by weight, calculated to the total composition.

Permanent shaping composition of the present invention can comprise further ceramide type of compound such as cetyl-PG-hydroxyethylpalmitamide.

Further optional ingredient are sterols, especially the phytosterols as preferred hair restructuring agents. Especially preferred ones are of plant origin for example ergosterol, sitosterol, stigmasterol, fucosterol, brassicasterol, fungisterol, campesterol, zymosterol, ascosterol, cerevisterol, episterol, faecosterol, spinasterol. Among those phytosterols, the ones found in “Avocadin” which is the unsaponified fraction of the avocado oil is more preferred.

Optionally fatty acids of C10 to C22 may be incorporated into the compositions of the present invention at a concentration of preferably 0.01 to 2.5% by weight calculated to total composition.

Another preferred compound in the permanent shaping composition of the present invention is silicone compounds and especially aminated silicones such as amodimethicone available from for example Dow Corning under the brand names Dow Corning 949 Emulsion and Dow Corning 2-8194. Concentration of silicones, especially amodimethicone, is in the range of 0.05 to 2.5%, preferably 0.1 to 1% by weight calculated to total composition.

Additionally, one or more natural oil component may be incorporated into the compositions of the present invention. Suitable are such as olive oil, almond oil, avocado oil, wheatgerm oil, ricinus oil or their mixture. Concentration of these natural oil ingredients should be 0.01 to 2.5%, preferably 0.01. to 1%, more preferably 0.05 to 0.5% by weight, calculated to total composition.

Further additional compounds may be present in the permanent shaping compositions of the present invention is ubichinone of the formula

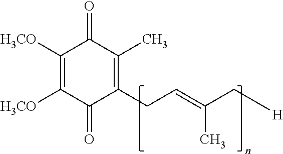

where n is a number between 1 and 10. Preferred ubichinones are the ones where n is a number between 6 and 10 and especially preferred is Ubichinone 50 where n is 10, also known as Coenzyme Q10. Concentration ubichinone of the above formula in permanent shaping compositions of the present invention is from 0.0001 to 1%, preferably from 0.0002 to 0.75%, more preferably from 0.0002 to 0.5% and most preferably from 0.0005 to 0.5% by weight, calculated to total composition.

In another preferred embodiment of the present invention, composition comprises one or more fatty alcohol with a chain length of 12 to 24 C atoms. Concentration of one or more fatty alcohol is in the range of 1 to 15%, preferably 1 to 10%, more preferably 2 to 7.5 and most preferably 2 to 5% by weight calculated to total composition.

Suitable non limiting examples are lauryl alcohol, myristyl alcohol, cetyl alcohol, stearyl alcohol cetearyl alcohol, behenyl alcohol and their mixtures. Preferred are cetyl alcohol, stearyl alcohol cetearyl alcohol, behenyl alcohol and their mixtures. Particularly preferred are cetyl alcohol, stearyl alcohol and their 1 to 1, by weight, mixture cetearyl alcohol.

The compositions used according to the invention can naturally comprise all the substances customarily found in permanent shaping compositions, a list of which will not be given here, and are preferably present as solutions, gels with a higher or lower viscosity, emulsions or creams. They can be single-phase products or compositions packed into separate packaging which are united upon application, as they are disclosed, for example, in DE-C 43 04 828.

In order to avoid repetition, reference is here made to the state of the art as it is described, for example, in “Ullmann's Encyclopedia of Industrial Chemistry”, Vol. A12 (1986), pages 588 to 591, and in particular to the monography of K. Schrader, “Grundlagen and Rezepturen der Kosmetika”, 2^{nd}. Ed. (1989, Huthig Buchverlag) pages 823 to 840, as well as the article by D. Hollenberg et. al. in “Seifen-Öle-Fette-Wachse”, 117 (1991), pages 81 to 87.

Composition of the present invention is used in a process for permanent waving wherein hair is washed or shampooed first and wound on the curlers, subsequently a reducing agent composition comprising at least one reducing agent, a first cationic polymer consisting of copolymer of vinylpyrrolidone and vinylimidazole and homopolymer of dimethyl diallyl ammonium chloride and a second cationic olymer selected fro urea copolymers is applied onto hair and after 1 to 45 min, preferably 1 to 30 min of processing time, depending on the hair strength, rinsed off from hair with tap water and an oxidizing composition comprising at least one oxidizing agent, preferably hydrogen peroxide or sodium bromate at a concentration of 0.5 to 10% by weight calculated to total of oxidizing composition, is applied onto hair and left on the hair 1 to 20 min and rinsed off and curlers removed from hair. In cases where additional conditioning composition is required this may as well be applied after finishing the process as described above.

In another process as described above the curlers are removed after rinsing off the reducing agent and before applying the oxidizing agent.

Further in another process, after rinsing off the reducing agent from hair, an intermediate treatment composition is applied onto hair and without rinsing off but after removing the excess amount of intermediate treatment with a towel, oxidizing composition is applied and at the end of the processing they are rinsed off from hair and curlers are removed from hair.

It has further been found out that the use of first cationic polymer consisting of a copolymer of vinylpyrrolidone and vinylimidazole and homopolymer of dimethyl diallyl ammonium chloride and a second cationic polymer selected from urea copolymers in the intermediate treatment composition improves the permanent shaping of hair as well in terms of curl appearance and natural look and feel of hair. Therefore, in another preferred form of the present invention, permanent shaping of hair is carried our with a process wherein hair is washed or shampooed first and subsequently a reducing agent comprising composition is applied onto hair and after 1 to 45 min, preferably 1 to 30 min of processing time, depending on the hair strength, rinsed off from hair with tap water and an intermediate treatment composition comprising a first cationic polymer consisting of a copolymer of vinylpyrrolidone and vinylimidazole and homopolymer of dimethyl diallyl ammonium chloride and a second cationic polymer selected from urea copolymers is applied and without rinsing off an oxidizing composition comprising at least one oxidizing agent, preferably hydrogen peroxide or sodium bromate at a concentration of 0.5 to 10% by weight calculated to total of oxidizing composition, is applied onto hair and left on the hair 1 to 20 min and rinsed off. In cases where additional conditioning composition is required this may as well be applied after finishing the above process. In case of permanent waving hair in the above process curlers are put onto hair before application of reducing composition and taken off from hair before application of oxidizing composition or after application and processing of the oxidizing composition.

The intermediate treatment composition has a pH value between 2.5 to 6, preferably 3 to 5.5 and most preferably 3 to 5.

A straightening process may also be carried out in a different process wherein hair is washed and/or shampooed and dried and reducing composition comprising at least one reducing compound and a first cationic polymer consisting of a copolymer of vinylpyrrolidone and vinylimidazole and homopolymer of dimethyl diallyl ammonium chloride and a second cationic polymer selected from urea copolymers is applied onto dry hair and processed for 5 to 60 min, preferably 5 to 45 min and rinsed off with water and dried and the dry hair physically straighten with hot iron at a temperature of 130 to 210° C. and subsequently an oxidizing composition comprising at least one oxidizing agent, preferably hydrogen peroxide or sodium bromate at a concentration of 0.5 to 10% by weight calculated to total of oxidizing composition, is applied onto hair and left on the hair 1 to 20 min and rinsed off.

The following examples are to illustrate the invention, but not to limit it.

### EXAMPLE 1

| | | |
|---|---|---|
| Ammonium thioglycolate (60%) | 21.3 | (% by wt. |
| Ammonium hydrogen carbonate | 5.0 | |
| 1,3-butylene gylcol | 3.0 | |
| Polyquaternium-87* | 0.25 | |
| Polyquaternium-2 | 0.25 | |
| PEG-40-Hydrogenated castor oil | 0.7 | |
| Perfume | 0.4 | |

| | |
|---|---|
| Ammonia, 25% | ad pH 8.3 |
| Water | ad 100.0 |

*Cationic polymer used was Luviquat Sensation from BASF.

With this composition the hair was permanently waved for about 15 minutes, rinsed and neutralized for about 8 minutes with a customary 2.5% H₂O₂ composition. Homogeneous wave appearance was obtained. Exclusion of Polyquaternium 87 and Polyquaternium 2 resulted in less homogeneous perm appearance.

It has furthermore found out that the improved permanent waving efficiency with the two cationic polymers in combination was synergistic as improved curling efficiency observed when both copolymers comprised in the compositions was not simply sum of the effects observed with the compositions comprising one of the cationic polymers at the same total polymer concentration.

In all of the following examples, Luviquat Sensation from BASF was used as the first cationic polymer. And Mirapol A-15 is used as the second cationic polymer.

### EXAMPLE 2

| | | |
|---|---|---|
| Ammonium thioglycolate (60%) | 20 | (% by wt. |
| Ammonium hydrogen carbonate | 4.0 | |
| 1,3-butylene gylcol | 3.0 | |
| Polyquaternium-87 | 0.25 | |
| Polyquaternium-2 | 0.25 | |
| PEG-40-Hydrogenated castor oil | 0.7 | |
| Perfume | 0.4 | |

| | |
|---|---|
| Ammonia, 25% | ad pH 8.3 |
| Water | ad 100.0 |

With this composition the hair was permanently waved for about 15 minutes, rinsed and neutralized for about 8 minutes with a customary 2.5% H₂O₂ composition. Homogeneous wave appearance was obtained. Exclusion of Polyquaternium-87 and Polyquaternium-2 resulted in less homogeneous perm appearance.

### EXAMPLE 3

### Alkaline Permanent Wave for Damaged Hair

| | | |
|---|---|---|
| Ammonium thioglycolate (60%) | 15.0 | (% by wt.) |
| Ammonium hydrogen carbonate | 2.5 | |
| Ceteth-20 | 0.7 | |
| 1,3-butylene gylcol | 0.5 | |
| Polyquaternium-87 | 0.5 | |
| Polyquaternium-2 | 0.5 | |
| Perfume | 0.4 | |

| | |
|---|---|
| Ammonia, 25% | ad pH 8.0 |
| Water | ad 100.0 |

The permanent wave achieved with this composition was similar to the one obtained with the composition according to Example 1.

Exclusion of the Polyquaternium-87 and Polyquaternium-2 led to waves with substantially weaker contours.

### EXAMPLE 4

| | | |
|---|---|---|
| Ammonium thioglycolate (60%) | 0.9 | (% by wt.) |
| Cystein hydrochloride | 5.7 | |
| Ammonium hydrogen carbonate | 1.5 | |
| Acetylcystein | 0.7 | |
| Cetrimonium chloride | 0.1 | |
| 1,3-butylene gylcol | 0.5 | |
| Polyquaternium-87 | 0.6 | |
| Polyquaternium-2 | 0.6 | |
| Amodimethicone | 0.2 | |
| Coenzyme Q10 | 0.05 | |
| Oleic acid | 0.05 | |
| Perfume | 0.4 | |

| | |
|---|---|
| Ammonia, 25% | ad pH 9.8 |
| Water | ad 100.0 |

The permanent wave achieved with this composition was similar to the one obtained with the composition according to Example 1.

Exclusion of the Polyquaternium-87 and Polyquaternium-2 led to substantially weaker waves.

### EXAMPLE 5

A permanent waving product consisting of two Compositions A and B, filled into a two-chamber packaging the chambers of which were kept separate until application, was prepared by destruction of the separating wall and applied onto human hair rolled onto curlers. The hair was rinsed after about fifteen minutes processing and neutralized for about five minutes with a 2.5% H₂O₂ neutralizer composition, rinsed again, shampooed and dried.

An expressive, even, intensive permanent wave was obtained.

An identical treatment which had no Polyquaternium-87 and Polyquaternium-2 showed a visibly inferior wave.

### Composition A:

| | | |
|---|---|---|
| Ammonium hydrogen carbonate | 4.5 | (g) |
| Polyquaternium-87 | 0.5 | |
| Polyquaternium-2 | 0.5 | |
| PEG-65-Hydrogenated castor oil | 0.8 | |
| Isopropyl alcohol | 1.5 | |
| Ethoxydiglycol | 2.0 | |
| Cocoamidopropyl betaine | 1.0 | |
| Perfume | 0.3 | |
| Coenzyme Q10 | 0.05 | |
| Turbidifying agent | 0.5 | |

| | |
|---|---|
| Ammonia, 25% | ad pH 8.4 |
| Water | ad 72.0 |

### Composition B:

| | | |
|---|---|---|
| Ammonium thioglycolate, 70% | 18.0 | (g) |
| Thiolactic acid | 2.0 | |
| 2-Methyl-1.3-propanediol | 0.5 | |

| | |
|---|---|
| Ammonia, 25% | ad pH 5.5 |
| Water | ad 28.0 |

After mixing of both Compositions, a ready-to-use product with a pH-value of 7.4 was obtained.

### EXAMPLE 6

A permanent waving product filled into a two-chamber package was prepared in analogy to Example 4:

### Composition A:

| | | |
|---|---|---|
| Ammonium hydrogen carbonate | 3.5 | (g) |
| Polyquaternium-87 | 0.5 | |
| Polyquaternium-2 | 0.5 | |
| Ethanol | 0.5 | |
| 1-Methoxypropanol | 1.0 | |
| Cocoamidopropyl betaine | 1.0 | |
| PEG-25-glyceryl cocoate | 0.8 | |
| Coenzyme Q10 | 0.1 | |
| Oleic acid | 0.05 | |
| Perfume | 0.3 | |
| Turbidifying agent | 0.5 | |

| | |
|---|---|
| Ammonia, 25% | ad pH 8.3 |
| Water | ad 72.0 |

### Composition B:

| | | |
|---|---|---|
| Ammonium thioglycolate, 70% | 13.0 | (g) |
| Thiolactic acid | 0.5 | |
| 2-Methyl-1.3-propanediol | 1.5 | |

| | |
|---|---|
| Ammonia, 25% | ad pH 5.5 |
| Water | ad 28.0 |

A product with a pH-value of 7.4 was obtained by mixing of the compositions immediately prior to application. After application onto dyed hair as described in Example 3, this mixture resulted in an expressive permanent wave, which had not effect whatever on the color gloss and color intensity.

### EXAMPLE 7

### Alkaline Permanent Waving Gel

| | | |
|---|---|---|
| Ammonium thioglycolate, 70% | 15.0 | (g) |
| Ammonium hydrogen carbonate | 4.5 | |
| PEG-40-Hydrogenated castor oil | 0.7 | |
| C₁₂-C₁₈-Fatty alcohol mixture | 3.5 | |
| Cetrimonium chloride | 2.0 | |
| Amodimethicone | 0.05 | |
| 2-Methyl-1.3-propanediol | 0.5 | |
| Polyquaternium-87 | 0.45 | |
| Polyquaternium-2 | 0.45 | |
| Coenzyme Q10 | 0.1 | |
| Perfume | 0.3 | |

| | |
|---|---|
| Ammonia, 25% | ad pH 8.0 |
| Water | ad 100.0 |

### Intermediate Treatment Composition

| | | |
|---|---|---|
| Asparagic acid | 0.25 | % by weight |
| Glutamic acid | 0.50 | |
| Alanin DL | 0.25 | |
| Magnesium sulfate | 10.00 | |
| Polyquaternium-87 | 0.25 | |

| | |
|---|---|
| Water | q.s. to 100 |

The above composition had a pH of 4.10.

### EXAMPLE 8

### Straightening Composition

| | |
|---|---|
| Thioglycolic acid | 8.0 (% by wt.) |
| C₁₆-C₂₂-Fatty alcohol mixture | 3.5 |
| Oleth-50 | 2.5 |
| Laureth-23 | 1.5 |
| Polyquaternium-87 | 0.5 |
| Polyquaternium-2 | 0.5 |
| Ethanol | 5.0 |
| Perfume | 0.6 |
| Monoethanolamine | ad pH 9.3 |
| Water | ad 100.0 |

1. Composition for the permanent shaping of human hair, comprising at least one reducing agent, a first cationic polymer consisting of copolymer of vinylpyrrolidone and vinylimidazole and a homopolymer of dimethyl diallyl ammonium chloride and a second cationic polymer selected from urea copolymers.

2. Composition according to claim 1, wherein the reducing agents are selected from thioglycolic acid, thiolactic acid and their salts, cystein and its hydrochloride salt, acetylcystein.

3. Composition according to claim 1, comprising reducing agent at a concentration of 0.5 to 15% by weight, calculated to total composition.

4. Composition according to claim 1, wherein the first cationic polymer is Polyquaternium-87 and it has a cationic charge density of between 3 and 4 meq/g at pH 7.0.

5. Composition according to claim 1, wherein the second cationic polymer is Polyquaternium-2.

6. Composition according to claim 1, wherein the first polymer is present at a concentration of 0.1 to 2.5%, by weight calculated to total composition.

7. Composition according to claim 1, wherein the second polymer is present at a concentration of 0.1 to 2.5 by weight calculated to total composition.

8. Composition according to claim 1, wherein the first and the second cationic polymers are comprised at a total concentration in the range of 0.2 to 5%, calculated to total composition, and at a weight ratio of the first to the second cationic polymer in the range of 5:1 to 1:5.

9. Composition according to claim 1, wherein the composition has a Brookfield viscosity of 1 to 50,000 mPa·s at 20° C.

10. Composition according to claim 1, further comprising at least one surfactant selected from anionic, nonionic, cationic and amphoteric surfactants at a concentration of 0.05 to 10% by weight, calculated to total composition.

11. Composition according claim 1, further comprising at least one fatty alcohol with a chain length of 12 to 24 C atoms, least one organic solvent, at least one ubichinone of the formula 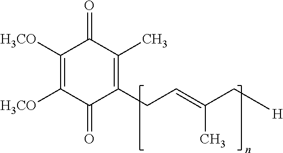
where n is a number between 1 and 10, at least one silicone compound, and at least one thickening agent.

12. Composition according to claim 1, wherein the composition has a pH in the range of 6.5 to 10.5.

13. (canceled)

14. (canceled)

15. (canceled)
